(19) United States
(12) Patent Application Publication
(10) Pub. No.: US 2021/0260151 A1
(43) Pub. Date: Aug. 26, 2021

(54) THERAPEUTIC COMPOSITION FOR ENDOMETRIOSIS SURGERY AND METHOD FOR USING THE SAME

(71) Applicant: TAMER AHMET SECKIN, NEW YORK, NY (US)

(72) Inventor: TAMER AHMET SECKIN, NEW YORK, NY (US)

(21) Appl. No.: 16/798,387

(22) Filed: Feb. 23, 2020

(51) Int. Cl. A61K 38/095; A61K 47/22; A61K 31/375; A61K 31/355; A61K 31/07; A61K 31/167; A61K 9/06; A61K 9/00

(52) U.S. Cl. CPC A61K 38/095; A61K 47/22; A61K 31/375; A61K 9/0019; A61K 31/07; A61K 31/167; A61K 9/06; A61K 31/355

(57) ABSTRACT

The present invention provides a therapeutic composition for administering during a surgery, especially an endometriosis surgery, comprising at least an active ingredient and an excipient, wherein the active ingredient comprises an antioxidant agent and the excipient comprises a semi-solid barrier. The present invention provides also a method for using such a therapeutic composition during a surgery.

### TECHNICAL FIELD

The present invention relates to a therapeutic composition for delivery during a surgery, especially an endometriosis surgery and a method for delivering such a therapeutic composition during a surgery.

### BACKGROUND ART

Recent studies have put the attention on the role of oxidative stress, defined as an imbalance between reactive oxygen species (ROS) and antioxidants, which may be implicated in the pathophysiology of endometriosis causing a general inflammatory response in the peritoneal cavity which may lead to pain and peritoneal adhesions. Further, bleeding during surgery, blood clots and fibrin deposits induce adhesions. Therefore, each surgery causes patient more adhesions of peritoneum and intra peritoneal organs. These adhesions may cause intestinal obstruction, pain and difficulties in repeated surgeries.

The haemostatic control of bleeding is very important for prevention of adhesions caused by blood clots and fibrin deposits. Conventional methods of administration of haemostatic agents in therapeutic doses frequently is associated with significant adverse side effects. Generally, vasopressin is administered by injection to the excision. However, injecting vasopressin to the patient may cause intraoperative cardiovascular complications. Especially, during a surgery with a plurality of excision sites, i.e. due to distributed endometrioma, controlling the dosage of injected vasopressin may be difficult.

U.S. patent application Ser. No. 16/798,371, which is incorporated here by reference, describes filling of an aqueous solution comprising Methylene Blue dye and Vasopressin into peritoneum in order to enhance the view during the laparoscopy and at the same time to prevent adhesions and to control oxidative stress in the peritoneum making use of therapeutic effects of Methylene Blue.

The additives in the aqueous solution prevent adhesion formation, oxidative stress and excessive bleeding by acting on the surfaces of all excised areas forming a preventive and protective barrier postoperatively. In some cases, an aqueous solution may be absorbed possibly faster than needed by the peritoneum not allowing a controlled delivery of a therapeutic. Further, in some surgeries, a therapeutic should act locally and for a predetermined time period and not be distributed to places outside of the peritoneal cavity to avoid side effects. However, solid barriers have limitations as a barrier, because the barriers are hard to apply to the complex geometries of the abdominal cavity.

There is a need for prevention of intestinal obstruction, pain and difficulties in repeated surgeries. Further, there is a need for prevention of adhesions due to a laparoscopic surgery while administering haemostatic agents in optimum dosage and in a retarded manner There is also a need for prevention of recurring of endometriosis after a laparoscopic surgery while controlling oxidative stress in the peritoneum.

### DISCLOSURE OF THE PRESENT INVENTION

The present invention provides a therapeutic composition for administering during a surgery, especially an endometriosis surgery, comprising at least an active ingredient and an excipient, wherein the active ingredient comprises an antioxidant agent and the excipient comprises a semi-solid barrier. The semi-solid barrier comprises a gel or a hydrogel, preferably a sprayable hydrogel or a supramolecular polymeric hydrogel. The therapeutic composition may further comprise a haemostatic agent. The antioxidant agent may comprise Methylene Blue. The therapeutic composition may comprise Vitamin C or one of the Vitamins C, E and A or a combination thereof as a further antioxidant agent. The haemostatic agent may comprise a local vasoconstructive agent or Vasopressin. The active ingredient may further comprise a local anesthetic. The local anesthetic may comprise Lidocain.

A therapeutic composition in form of gel or hydrogel does not have drawbacks of conventional delivery methods.

In a further aspect, the present invention provides a method for administering a therapeutic composition during a surgery comprising excising a site and covering the site of excision with the therapeutic composition. The method may comprise covering the site of excision with the therapeutic composition by injecting or spraying the therapeutic composition onto the site of excision.

The additives in the therapeutic composition of the present invention prevent adhesion formation, oxidative stress and excessive bleeding by acting on the surfaces of all excised areas forming a preventive and protective barrier postoperatively.

The therapeutic composition of the present invention may be used in all types of surgeries including all abdominal and thoracic surgeries on behalf of its antioxidant, adhesion preventive and bleeding control manner.

These and other objects of the present invention will be apparent from the drawings, claims and detailed descriptions herein.

### BRIEF DESCRIPTION OF THE DRAWINGS

The accompanying drawings, referred to herein and constituting a part hereof, illustrate preferred embodiments of the present invention and together with the description, serve to explain the principles of the present invention, wherein:

FIG. 1 is a partial cross-sectional side view of a peritoneum of a patient schematically showing an excision site before removal of endometriosis;

FIG. 2 is a partial cross-sectional side view of a peritoneum schematically showing an excision site after removal of endometriosis;

FIG. 3 is a partial cross-sectional side view of a peritoneum schematically showing an embodiment of the present invention administered onto an excision site of endometriosis.

### DESCRIPTION OF AN EXEMPLARY EMBODIMENT

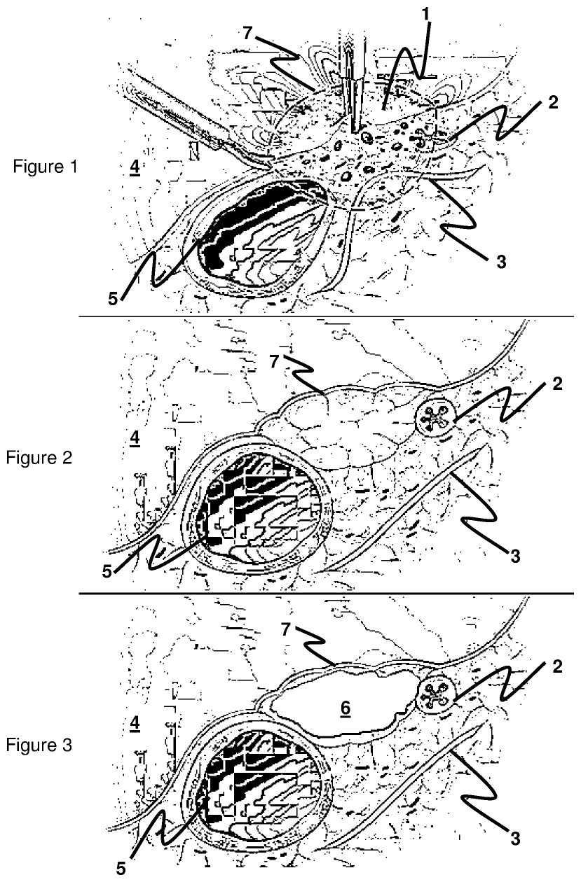

Referring to FIG. 1, a body tissue in the peritoneum (4) of a female body is illustrated in cross-sectional view before excision removal of endometriosis, showing an endometriosis (1), and contracted ureter (2), nerve (3), rectum (5) because of the developed endometriosis. FIG. 1 shows also an excision site (7) and surgical tools for removal of endometriosis (1).

FIG. 2 shows that the contraction of ureter (2), nerve (3) and rectum (5) is released after excision removal of endometriosis leaving an open excision site (7).

FIG. 3 shows the excision site (7) of FIG. 2 now covered with the inventive therapeutic composition comprising a semi-solid barrier as a vehicle and excipient for retarded release. According to this embodiment, the semi-solid barrier was formed as hydrogel, which was then sprayed onto the excision site (7) so as to cover the excision site (7). The surgery was then completed leaving the excision site (7) covered with the therapeutic composition. Alternatively, the inventive composition may have been injected instead of sprayed onto the excision site (7).

While the above description contains many specifics, these specifics should not be construed as limitations on the scope of the invention, but merely as exemplifications of preferred embodiments thereof. Those skilled in the art will envision many other possible variations that are within the scope and spirit of the invention as defined by the claims appended hereto.

1. Therapeutic composition for administering during a surgery, especially an endometriosis surgery, comprising at least an active ingredient and an excipient, wherein said active ingredient comprises an antioxidant agent and said excipient comprises a semi-solid barrier.

2. Therapeutic composition according to claim 1, wherein said semi-solid barrier is formed as gel or hydrogel or sprayable hydrogel or supramolecular polymeric hydrogel.

3. Therapeutic composition according to claim 1, further comprising a haemostatic agent.

4. Therapeutic composition according to claim 1, wherein said antioxidant agent comprises Methylene Blue.

5. Therapeutic composition according to claim 1, further comprising Vitamin C or one of the Vitamins C, E and A or a combination thereof.

6. Therapeutic composition according to claim 1, wherein said haemostatic agent comprises a local vasoconstructive agent or Vasopressin.

7. Therapeutic composition according to claim 1, wherein said active ingredient further comprises a local anesthetic or Lidocain.

8. Method for administering a therapeutic composition during a surgery, especially an endometriosis surgery, comprising excising a site and covering said site of excision with a therapeutic composition comprising at least an active ingredient and an excipient, wherein said active ingredient comprises an antioxidant agent and said excipient comprises a semi-solid barrier.

9. Method according to claim 8, wherein covering said site of excision with said therapeutic composition comprises injecting or spraying said therapeutic composition onto said site of excision.

10. Method according to claim 8, wherein said site of excision is left covered with said therapeutic composition after the surgery.
